SEQUENCE LISTING (8 sequences)

SEQ ID NO: 1 - DNA, 17 bp, Artificial Sequence
  Description of Artificial Sequence PNA construct
tcaacgttagcttcacc

SEQ ID NO: 2 - DNA, 17 bp, Artificial Sequence
  Description of Artificial Sequence PNA construct
ttaacgctagctttacc

SEQ ID NO: 3 - protein, 7 aa, Artificial Sequence
  Description of Artificial Sequence Nuclear Localization Signal peptide
PKKKRKV

SEQ ID NO: 4 - DNA, 17 bp, Artificial Sequence
  Description of Artificial Sequence PNA sequence
cagctggaattcggggc

SEQ ID NO: 5 - DNA, 17 bp, Artificial Sequence
  Description of Artificial Sequence PNA sequence
cggggcttaaggtcgac

SEQ ID NO: 6 - DNA, 16 bp, Artificial Sequence
  Description of Artificial Sequence PNA sequence
ccgtccaagacctacc

SEQ ID NO: 7 - DNA, 15 bp, Artificial Sequence
  Description of Artificial Sequence PNA sequence
ccatgttttgccatt

SEQ ID NO: 8 - DNA, 16 bp, Artificial Sequence
  Description of Artificial Sequence PNA sequence
gacagtgtcacacatt

(19) United States
(12) Patent Application Publication
(10) Pub. No.: US 2005/0256051 A1
(43) Pub. Date: Nov. 17, 2005

(54) Nuclear receptor-mediated introduction of a PNA into cell nuclei

(75) Inventor: Patricia L. Morris, Woodside, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: The Population Council, Inc., New York, NY (US)

(21) Appl. No.: 11/167,616

(22) Filed: Jun. 27, 2005

Related U.S. Application Data
(63) Continuation of application No. 10/122,973, filed on Apr. 12, 2002, now abandoned.
(60) Provisional application No. 60/284,658, filed on Apr. 13, 2001.

(51) Int. Cl. A61K 48/00; C07K 14/775

(52) U.S. Cl. 514/12; 530/359

(57) ABSTRACT

Disclosed are compositions and methods for introducing Peptide Nucleic Acids (PNAs) into cell nuclei. The PNAs are linked to a ligand that binds a nuclear receptor. The methods have therapeutic and diagnostic applications.

### CROSS-REFERENCE TO RELATED APPLICATIONS

This application is a continuation of U.S. application Ser. No. 10/122,973, filed on Apr. 12, 2002, which claims the benefit of the filing date of U.S. Provisional Application No. 60/284,658, filed Apr. 13, 2001, the disclosure of which is hereby incorporated herein by reference.

### STATEMENT REGARDING FEDERALLY SPONSORED RESEARCH

The research for the present invention was supported by National Institute of Health grants HD-29428 and HD-13541. Therefore the United States government may have certain rights in this invention.

### BACKGROUND OF THE INVENTION

Peptide nucleic acids (PNAs) are synthetic structural homologues of nucleic acids in which the negatively charged phosphate-sugar backbone of the polynucleotide is replaced by an uncharged polyamide backbone consisting of achiral N-2-aminoethyl glycine units. Each unit is linked to a purine or pyrimidine base to create the specific sequence required for hybridization to the targeted polynucleotide (1,2). There are numerous advantages of PNAs in anti-gene or antisense applications to down-regulate transcription or translation in living cells. The absence of a negatively charged backbone facilitates PNA invasion of the DNA double helix to form a stable PNA-DNA hybrid with high mismatch discrimination (3,4). This interaction is further stabilized in chromatin of live cells by two important observations: it has been demonstrated that in the cellular environment PNA-DNA hybrids are more stable than their homologues DNA-DNA since they are ionic strength independent (5); PNA binding to supercoiled DNA is stronger than to linear DNA and favored in transcriptionally active chromatin (6,7). The great stability of PNA-DNA hybrids in chromatin has made it possible to isolate the hybrids as components of restriction fragments as large as 23 kb (8). Moreover, the unusual chemical structure of PNAs makes them highly resistant to both nucleases and proteases (9). Basically, compared to DNA antisense molecules, PNAs bind more tightly to their target; they are less tolerant of base mismatches so they do not bind DNAs that have similar sequences to the target; and they tend not to degrade en route to the target.

Previous experiments with permeabilized cells and isolated nuclei (10,7), as well as experiments conducted in vivo (11), have shown that complex sequence PNAs are highly effective in blocking transcription of the targeted gene without inhibiting RNA synthesis in unrelated genes. It has also been shown that PNA binding effectively blocks transcription of the c-myc gene in both directions (7). Still, a major problem in the application of PNAs as anti-gene agents is that they show restricted ability to penetrate the nuclei of cells in culture or in vivo (11,12).

There are indications that antisense or anti-gene PNAs, if artificially allowed to enter the nucleus, can inhibit translation/transcription (7,10,13). Recent trials of a few vectors have also shown successful delivery of the fused PNAs to the nuclei of live cells (11,14-17). In these techniques, however, the PNAs did not discriminate target cells from non-target cells i.e., without regard to critical cell function. In this regard, U.S. Pat. No. 6,180,767 B1 teaches compositions and methods for targeting PNAs to specific cells by linking the PNA to a ligand that is capable of binding a cell surface receptor. Cell surface receptors are not constant fixtures of the cell membrane, however. In addition, the vast majority of ligands that bind cell surface receptors are peptides, and thus are susceptible to degradation before they even bind the receptor.

Thus, there remains a need for compositions and methods that facilitate entry of PNAs into specific cell nuclei.

### SUMMARY OF THE INVENTION

Applicant has invented compositions and methods for introducing PNAs into cell nuclei by targeting the PNAs to nuclear receptors. Unlike cell surface receptors, nuclear receptors are intracellular receptors. Thus, one aspect of the present invention is directed to a composition of matter comprising a PNA linked (or coupled) to a ligand that binds a nuclear receptor (e.g., a conjugate of a ligand and a PNA, or a ligand/PNA conjugate). Since nuclear receptors are expressed selectively in different types of cells, the compositions of the present invention can be constructed to be targeted to specific cells or cell types. The nuclear receptor becomes activated when bound to the ligand. Once activated, the receptor and the PNA translocate across the nuclear membrane. Thus, the ligand that is linked to the PNA activates the receptor resulting in relatively efficient and selective internalization or uptake of the PNA into cell nuclei.

In some preferred embodiments, the PNA binds an oncogene or portion thereof. In other preferred embodiments, the ligand binds an androgen receptor such that the PNA is delivered into the nucleus of a cell that expresses an androgen receptor. Methods of making the compositions by linking the ligand to the PNA, are also provided.

Another aspect of the present invention is directed to a method for introducing the compositions into cell nuclei by contacting the compositions with the cells. In embodiments wherein the PNA acts in an antisense fashion, the method is directed to inhibiting transcription of a gene in the nucleus of a cell. In these embodiments, the PNA functions primarily by binding the target genomic DNA and inhibiting transcription, and thus translation. In preferred embodiments, the inhibition of transcription occurs in vivo, whereby the composition is administered to an organism such as a human subject. In other preferred embodiments, the PNA targets an oncogene and the human is a cancer patient.

Even though the compositions of the present invention penetrate cell membranes rather indiscriminately, uptake of the PNA into the cell nucleus occurs only if the cell contains the nuclear receptor for the ligand. In those cells that do not contain the nuclear receptor, the PNA is inactive (e.g., it does not bind complementary nucleic acid) and is eventually degraded by enzymes in the cell cytoplasm. The present invention enhances the selectivity and therapeutic effectiveness of specific PNA anti-gene therapy in living cells. The efficiency of PNA uptake into the nucleus can be orders of magnitude greater than other techniques, and the PNA is stable once it is in the nucleus.

In yet another aspect of the present invention, the compositions of the present invention also contain one or more detectable labels and are used diagnostically e.g., to detect excess copies of a pathogenic gene in a surgical biopsy. In preferred embodiments, the label is a fluorescent dye.

### BRIEF DESCRIPTION OF THE DRAWINGS

FIG. 1 is a chemical formula of a composition of the present invention.

FIGS. 2A-2H are photomicrographs that illustrate the cellular localization of PNAs in LNCaP and DU145 cells.

FIGS. 3A-D are graphs that illustrate the effects of PNAs on cell number (FIGS. 3A and C) and cell viability (FIGS. 3B and D) for each of LNCaP and DU145 cell lines respectively.

FIG. 4A contains two graphs and FIG. 4B contains 4 Western blots, illustrating the effect of the different specific PNA constructs on c-myc expression in each of LNCaP and DU145 cell lines.

### DETAILED DESCRIPTION

Intracellular or nuclear receptors are ligand-dependent transcription factors that respond to a variety of lipophilic endocrine and dietary-derived factors. Distinct receptors are expressed selectively in both cell-and tissue-specific manners. Molecular mechanisms for these intracellular regulators include ligand-binding, nuclear translocation and membrane trafficking and effects of transcription. Ligand-dependent and independent mechanisms alike regulate the activation and deactivation of the various nuclear receptors both within the cytoplasm and nucleus of distinct receptor-positive cells. Nuclear receptors include many families of receptors e.g., intracellular steroid receptors, orphan nuclear receptors, nuclear hormone receptors and vitamin receptors. Examples of nuclear receptors and corresponding natural ligands or synthetic activators include PPARs (peroxisome proliferator activated receptors, and for its gamma isomer, 15-deoxy-Δ^{12,14}-prostaglandin J₂), RXRs (retinoic acid receptors, and for the α, β and γ isomers, 9-cis-retinoic acid), FXRs (farnesoid X receptors, and retinoic acid and TTNPB), LXRs (liver X receptors, and for the α isomer, 24-OH-cholesterol), BXRs (benzoate X receptors, and 4-amino-butyl benzoate), Carβ (constitutive androstane receptor, and androstanol) PXRs (pregnane X receptors, and pregnenolone-16 carbonitrile) and SXRs (steroid and xenobiotic receptors, and rifampicin). See, Blumberg, et al., Genes & Development 12:3149-3155 (1998); Evans, Science 240(4854):889-895 (May 13, 1988); Mangelsdorf, et al., Cell 83(6):841-850 (1995); and McKenna, et al., J. Steroid Biochem. Mol. Biol. 74(5):351-356 (2000). PXRs, SXRs and CARs are highly expressed in the liver and respond to steroidal ligands. PXRs are activated by synthetic C21 steroids. SXRs are expressed at high levels in liver and intestine, both sites of steroid and xenobiotics metabolism, and are activated by a diverse group of steroid agonists and antagonists including estranes, androstanes and pregnanes. PPARs, particularly the gamma isomer, are expressed in several cell types including mammary epithelia, colonic epithelia and in two different classes of macrophages. These receptors promote cell differentiation in breast cancer and liposarcoma cell lines. The LXRα isomer has enriched expression in the liver whereas the β isomer is expressed ubiquitously. LXRs are activated by oxidized cholesterol derivatives such as 22(R) and 24(S) hydroxycholesterol and 24(S), 25-epoxycholesterol. Many high affinity synthetic analogs of natural ligands for steroid and thyroid hormone receptors have been developed. See, Krieger, in Endrocrinology and Metabolism, Felig, et al. Eds. (McGraw-Hill, New York, 1981), pp. 125-149. There are several thyroid hormone receptors. Weinberger, et al., Nature (London) 324, 641 (1986); Benbrook, et al., Science 230, 788 (1987). At least one such receptor is preferentially expressed in neurons. Thompson, et al., Science 237, 1610 (1987).

The compositions of the present invention are designed taking into account the type of cell that expresses the target nucleic acid, and the nuclear receptor(s) that is/are expressed in it. Androgen receptors, for example, are expressed in prostate cells. Thus, PNA/androgen conjugates are useful in targeting nucleic acids in these cells to treat diseases such as prostate cancer and BPH (benign prostatic hypertrophy). Conjugates containing estrogens as a ligand bind estrogen receptors expressed in breast, ovarian and uterine cells, and thus are useful in treating diseases that affect these cells such as breast, ovarian and uterine cancer. Conjugates containing retinoids as a ligand bind retinoid receptors present in skin cells, and thus are useful in treating dermatological disorders e.g., dermatological cancers. Conjugates containing a progestin (e.g., progesterone and derivatives thereof e.g., medroxyprogesterone, and agonists and antagonists of mifepristone and 19-nortestosterone derivatives) as the ligand bind progesterone receptors present in uterine, cervical and mammary cells, and thus are useful in treating diseases of the female reproductive tract, including endometrial disease, metastatic breast cancer and cervical cancer. Conjugates containing a glucocorticoid bind glucocorticoid receptors present in tumor cells, and thus are useful in treating Cushing's disease and lymphomas. Conjugates containing a T3 or T4 as a ligand bind thyroid hormone receptors present in thyroid cells and thus are useful in treating thyroid cancer and thyroiditis. Compositions containing a mineralocortoid as a ligand bind mineralocorticoid receptors present in kidney and adrenal cells, and thus are useful in treating renal and adrenal diseases. Conjugates containing a steroid (e.g., cholesterol and sterols) as a ligand bind specific nuclear receptors present in select hormone-dependent or ligand-dependent cells, and thus are useful in treating prostatic, breast and uterine cancers, and BPH.

As described herein, ligands that bind a given nuclear receptor are known in the art or may be selected in accordance with standard techniques. The ligands may be designed based on naturally or non-naturally occurring compounds. Yet other examples of ligands that bind androgen receptors include testosterone and testosterone derivatives such as dihydrotestosterone, non-5α-reducible androgens including 7α-modified-androgens e.g., 7α-alkyl-androgens such as 7α-methyl-14-dehydro-19-nortestosterone, 7α-methyl-17a,β-propionyloxy-D-homoestra-4,16, dien-3-one and 7α-methyl-19-nortestosterone (MENT), and testosterone derivatives having a non-hydrogen substitution in the 6α or 7α position e.g., 7-α-methyl testosterone, 7-α-methyl-11β-hydroxytestosterone, 7-α,17-dimethyltestosterone, 7-α,17-dimethyl-11β-hydroxytestosterone, 7-α,17-dimethyl-19-nortestosterone, 7-α,17-dimethyl-11β-hydroxy-19-nortestosterone, 6-α-methyl testosterone, 6-α-methyl-19-nortestosterone, 6-α-methyl-11β-hydroxytestosterone, 6-α,17-dimethyltestosterone, 6-α,17-dimethyl-11β-hydroxytestosterone, 6-α,17-dimethyl-19-nortestosterone and 6-α,17-dimethyl-11β-hydroxy-19-nortestosterone). Ligands that bind other non-androgen hormone receptors such as estrogens include estrogen and estrogen derivatives such as 17-beta estradiol and estriol. Progesterone receptors, retinoid receptors, thyroid hormone receptors and vitamin receptors, described above, are further examples of non-androgen nuclear hormone receptors that may also be targeted in accordance with the present invention. Sterols and orphan nuclear receptors (which may be targeted with xenobiotics as described in Xie et al., J. Biol. Chem. 276:37739-42 (2002)) as described above are examples of non-hormone nuclear receptors that can be targeted. Standard techniques for identifying further ligands include structure/activity assays and binding assays. The majority of ligands useful in the present invention are non-peptides.

Methods for the preparation of peptide nucleic acids are described in the following, the entire disclosures of which are incorporated herein by reference: International Patent Applications PCT/EP92/01219 (WO 92/20702), PCT/EP92/01220 (WO 92/20703), PCT/IB94/00142 (WO 94/25477), PCT/US94/06620 (WO 94/28720), PCT/US94/07319 (WO 95/01370), and PCT/US94/08465 (WO 95/03833). Essentially, PNAs are synthesized by adaptation of solution or solid phase peptide synthesis procedures. PNA synthesis is reviewed in PCT/US94/08465, page 11, line 6-page 23, line 7. The PNAs may be synthesized inexpensively on a large scale. PNAs may be synthesized by either solution phase or solid phase methods adapted from peptide synthesis. For example, PNAs can be synthesized from four protected monomers containing thymidine, cytosine, adenine and guanine via solid-phase peptide synthesis, by a modification of the Merrifield method (Merrifield, J. Am. Chem. Soc. 85:2149-2154 (1963); Merrifield, Science 232:341-347 (1986)) employing, for example, BOC-Z protected monomers (Christensen et al., J. Peptide Science 3:175-183 (1995)).

In general, the PNA portion of the conjugates of the present invention ranges from about 8 to about 60 subunits in length. In other embodiments, the PNAs range from about 10 to about 30 subunits in length. In still other embodiments of the present invention PNAs may range in size from about 12 to about 25 or 26 subunits in length. In yet further embodiments of the present invention, PNAs may range in size from about 12 to about 20 subunits in length.

The ligand is attached to the PNA by chemical coupling techniques. The choice of the attachment site on the PNA depends on the mode of interaction of the ligand with its receptor and the chemical nature of the ligand. Preferably, the ligand is attached to either terminal subunit of the PNA, although conjugation to an internal subunit is not excluded.

The ligand molecule may be attached directly to the PNA. Alternatively, the two compounds are coupled in a spaced relation, through inclusion of a linker moiety. Linkers act as bridging moieties and provide flexibility and unhindered steric access to the nuclear receptor. Thus, they reduce the steric effects of the molecular bulk of the PNA and its proximity to the ligand. The linker comprises any chemical group that is compatible with the ligand and PNA and which does not adversely affect either conjugate uptake or PNA hybridization to the target nucleic acid. Preferred linkers include NH₂-8-amino-3,6 dioxa-octanoyl-acid and NH₂-8-amino-caprylic-acid in a NH-Fmoc form. The length of the linking moiety varies depending upon the specific PNA and ligand. Preferably, the ligand is separated from the PNA by a distance of from about 10 to about 30 angstroms. Linker moieties are selected accordingly. In addition to selecting a chemically distinct moiety, the length of the linker can be increased, for example, by using 2 or more such moieties.

The compositions of the present invention may be used as therapeutic agents to ameliorate, arrest or prevent abnormal cell development on the cellular level, kill, cause growth arrest or inactivate animal (e.g., mammalian) cells that contribute to the development or progression of disease. In preferred embodiments, the PNA has a sequence complementary to a gene or portion thereof that causes or mediates malignancy (i.e., an oncogene) or a non-malignant disease characterized by abnormal cell growth, proliferation or hypertrophy of tissue. PNAs that target such genes are known in the art or are designed in accordance with standard techniques based on the sequence of the target nucleic acid. The nucleic acid sequences targeted for PNA binding according to the practice of the present invention may comprise, for example, oncogene or proto-oncogene genomic DNA (through triplex formation) or mRNA (through duplex formation). For example, c-myc expression may be targeted for inhibition, for treatment of hematological, mammary (e.g., breast) and colorectal malignancies (Gazin et al., EMBO J. 3:383-387 (1984)). Ki-ras may be targeted for treatment of pancreatic, colorectal and pulmonary malignancies (Shimizu et al., Nature 304:497-500 (1983)). Inhibition of c-myb expression is useful in the treatment of leukemias (U.S. Pat. No. 5,098,890), colorectal carcinoma (PCT/US92/04318) and melanoma (PCT/US92/09656). Expression of the hybrid oncogene bcr-abl may be targeted for treatment of Philadelphia chromosome-positive leukemias (PCT/US92/05035). Other oncogene and proto-oncogene targets for expression inhibition are known to those skilled in the art. At the DNA level, the irreversible anti-gene effect of an antisense PNA shuts down the expression of a particular gene. For instance, there is increasing evidence (7,11,13-15, 23) that PNAs can bind their complementary sequences in chromatin with high specificity, thereby effectively blocking transcription and translation.

The PNAs may be designed to target other genes having functions unrelated to regulation of cell growth and proliferation. For example, genes encoding prostaglandins, cytokines (e.g., interleukins such as IL-1 beta and IL-6), or tumor necrosis factor may be targeted to control inflammation; genes encoding angiotensin II or acetylcholinesterase may be targeted to control hypertension; genes encoding heme oxygenase (HO) and nitric oxide synthase (NOS) may be targeted to control neurological disorders such as neuromuscular dystrophies and Alzheimer's disease; and genes encoding lipid binding proteins having SMART (steroidogenic acute regulatory protein related (StAR) lipid transfer domains) domains e.g., StaR (steroidogenic acute regulatory protein), can be targeted to control various metabolic disorders.

The conjugates of the present invention may also be useful in the treatment of viral infections. Targets for treatment of viral infection include nucleic acids of human immunodeficiency virus (Ratner et al., Nature 313:277-284, 1985), herpes simplex virus (Smith et al., Proc. Natl. Acad. Sci. USA 83:2787-2791, 1986)), influenza virus (Leiter et al., Proc. Natl. Acad. Sci. USA 87:3430-3434 (1990)) and rabies virus.

The conjugates of the present invention may also find utility in the treatment of autoimmune disorders. Inadvertent production of antibodies against normal body tissues and structures results in degeneration of the target tissue (Davis, Annul. Rev. Biochem. 59:475-496 (1990)). Conjugates comprising PNA complementary to unique sequences in the autoimmune B-cell immunoglobulin genes or T-cell receptor genes may be capable of suppressing production of autoimmune antibodies or receptors by the particular plasma cell clonal lines involved. This approach may be of value in treating arthritis, systemic lupus erythromatosus, and myasthenia gravis, among other autoimmune disorders. PNA oligomer therapy may also be of value in suppressing the graft rejection response without compromising an individual's entire immune system.

The conjugates of the present invention may also be useful in the treatment of endocrinological disorders. Targeting of human growth hormone expression for inhibition by PNA oligomers is a potential treatment for acromegaly. Neurological diseases such as Alzheimer's disease may be treatable using conjugates comprising PNA oligomers targeting mutant beta-amyloid protein expression. It has been suggested that the monoamine oxidases may play a role in some forms of mental illness. The cDNAs for the A and B forms of monoamine oxidase have been isolated and cloned (Bach et al., Proc. Natl. Acad. Sci. USA 85:4934-4938 (1988)). Expression of theses genes may be useful targets for inhibition by complementary PNA oligomers.

For therapeutic or prophylactic treatment, the conjugates of the invention can be formulated in a pharmaceutical composition, which may include carriers, thickeners, diluents, buffers, preservatives, surface active agents and the like. Pharmaceutical compositions may also include one or more active ingredients such as antimicrobial agents, anti-inflammatory agents, anesthetics, and the like in addition to the ligand/PNA conjugate.

The pharmaceutical composition may be administered in a number of ways depending on the nature of the disease, whether local or systemic treatment is desired, and on the area to be treated. Administration may be performed topically (including ophthalmically, vaginally, rectally, transdermally, intranasally), orally, by inhalation, or parenterally, for example by intravenous infusion, drip or injection, or subcutaneous, intraperitoneal or intramuscular injection. Intravenous administration is utilized for rapid systemic distribution. =p Formulations for topical administration may include ointments, lotions, creams, gels, drops, suppositories, sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets, or tablets. Thickeners, flavorings, diluents, emulsifiers, dispersing aids or binders may be desirable. Formulations for parenteral administration may include sterile aqueous solutions that may also contain buffers, diluents and other suitable additives.

Preferred treatment regimens may include daily slow infusion, daily subcutaneous injection, daily transdermal patch wearing, daily nasal atomizer spray, weekly intramuscular injection, or monthly subcutaneous depot. The ligand/PNA conjugate may be delivered via slow release pledget placed for subcutaneous, intramuscular, intra-tumoral or intracranial release. Ideally, a slow release pledget may be used in place of a debulked tumor, for adjuvant therapy. See, for example, Brem et al., Lancet 345: 1008-1012 (1995). In some therapeutic indications that involve localized neoplasia, benign hypertrophy or growth, local administration is provided e.g.; by dermal patch delivery technologies, subcutaneous implants, or tissue-seed implants. For fertility regulation, therapeutic applications in females may include delivery of nontoxic sustained release forms by vaginal or uterine inserts such as rings. Drug delivery in males may require subdermal implants. Further modes of administration include the delivery of the drug in cream or jelly preparations. Systemic (e.g., parenteral) delivery of the vector-anti-gene peptide nucleic acids may be required for treatment of more advanced forms of cancer.

Dosing is dependent on severity and responsiveness of the condition to be treated, but will normally be one or more doses per day, with course of treatment lasting from several days to several months or until a cure is effected or a diminution of disease state is achieved. Persons of ordinary skill can easily determine optimum dosages, dosing methodologies and repetition rates. A dosage of from about 0.1 to about 3.0 mg/kg/day, more preferably from about 0.1 to about 1.0 mg/kg/day, is believed useful, based upon animal experiments where antisense DNA phosphorothioates were effective in animals in a subcutaneous/intraperitoneal dosage of 5-50 mg/kg/day.

Therapeutic end points can be determined by ablation of target gene expression (e.g., by Northern hybridization or PCR for detection of relevant mRNA, or Western blotting for detection of the relevant gene product), or by ablation of tumor load, viral load or disease symptoms.

Treatments of this type can be practiced on a variety of organisms ranging from unicellular prokaryotic and eukaryotic organisms to multicellular eukaryotic organisms, including humans. Any organism that utilizes DNA-RNA transcription or RNA-protein translation as a fundamental part of its hereditary, metabolic or cellular control is susceptible to therapeutic and/or prophylactic treatment in accordance with the invention. Seemingly diverse organisms such as bacteria, yeast, protozoa, algae, insects, all plants and all higher animal forms, including warm-blooded animals, can be treated, provided that an equivalent intracellular or nuclear receptor is present. Further, each cell of multicellular eukaryotes can be treated since they include both DNA-RNA transcription and RNA-protein translation as integral parts of their cellular activity. Furthermore, many of the organelles (e.g., mitochondria and chloroplasts) of eukaryotic cells also include transcription and translation mechanisms. Thus, single cells, cellular populations or organelles can also be included within the definition of organisms that can be treated with therapeutic or diagnostic phosphorothioate oligonucleotides. As used herein, therapeutics is meant to include the eradication of a disease state, by killing an organism or by control of erratic or harmful cellular growth or expression.

The compositions of the present invention can further contain a detectable label and be used for diagnostic applications. In preferred embodiments, the label is a fluorescent dye or a fluorophore such as ethidium. It is preferred that the label is attached to one end of the PNA and a receptor ligand attached to the other. Diagnostic applications include probing for excess copies of a pathogenic gene in cells obtained from a biopsy. Following uptake of the labeled conjugate by the cells, PNA hybridization with a target nucleic acid sequence results in introduction (e.g., intercalation) of the label into adjacent nucleic acid, elevating the quantum yield of the signal, facilitating scoring e.g., by flow cytometry.

The invention will be further described by reference to the following experimental work. This section is provided for the purpose of illustration only, and is not intended to be limiting unless otherwise specified. In this regard, the invention clearly includes ligands other than those that bind to an androgen receptor, and/or PNAs that are complementary to (or hybridize with) a sequence of a gene other than a c-myc gene. This work is also described in Boffa, et al., Cancer Res. 60:2258-2262 (2000), the disclosure of which is hereby incorporated by reference in its entirety.

A Model for Testing Dihydrotestosterone, as a Cell-Specific Vector for Cancer Therapy

In this example, dihydrotestosterone (T) was covalently linked to a PNA to form a PNA-dihydrotestosterone complex, in which the dihydrotestosterone acts as a vector for targeting c-myc DNA to prostatic cancer cell nuclei of LNCaP cells, which express Androgen Receptor (AR) gene, and DU145 cells, in which the AR gene is silent. Dihydrotestosterone was covalently linked to the N-terminal position of a PNA complementary to a unique sequence of c-myc oncogene (PNAmyc-T). To localize PNAmyc-T and vector-free PNA within the cells, a Rhodamine (R) group was attached at the C-terminal position (PNAmyc-R, PNAmyc-TR); cellular uptake was monitored by confocal fluorescence microscopy. PNAmyc-R was detected only in the cytoplasm of both prostatic cell lines, whereas PNAmyc-TR was localized in nuclei as well as in cytoplasm of LNCaP cells. In contrast, PNAmyc-TR uptake in DU145 cells was minimal and exclusively cytoplasmic. In LNCaP cells, MYC protein remained unchanged by exposure to vector-free PNAmyc, while a significant and persistent decrease was induced by PNAmyc-T. In DU145 cells, MYC expression was unaltered by PNAmyc with or without T vector. The data show that T vector facilitated cell-selective nuclear localization of PNA and its consequent inhibition of c-myc expression.

PNA Design

A set of nine related PNA constructs as shown in Table 1 were used to study the role of the T vector in directing their intracellular localization and their effects on c-myc expression. PNAmyc_{wt} represents a unique sequence of c-myc (Accession number X00364) (21) located in the second exon of the oncogene, i.e. bases 4528-4544 having the sequence of TCA ACG TTA GCT TCA CC (SEQ ID NO:1). SEQ ID NO:1 was tested with and without the dihydrotestosterone (T) vector, covalently linked in the N-terminal position (PNAmyc_{wt}-T; Table 1).

In the first set of experiments, two PNAs were tagged at their C-terminal end with Rhodamine (R) to allow their localization within the cell by fluorescence/phase contrast confocal microscopy. A rhodamine tag was also used to test the specificity of a PNA construct (PNA-mycₘᵤₜ-R) containing a three-base substitution in the c-myc sequence thus leaving the purine/pyrimidine ratio unchanged. A mutant of the PNA represented by SEQ ID NO:1 has the sequence of TTA ACG CTA GCT TTA CC (SEQ ID NO:2). Constructs containing this mutant sequence were used as negative controls.

TABLE 1

Composition of the PNA constructs with their
corresponding abbreviations

| | | | | |
|---|---|---|---|---|
| | | C- | | N- |
| Description | Abbreviation | terminal | PNA sequence | terminal |

| | | | | | |
|---|---|---|---|---|---|
| c-myc anti-gene | PNA-myc_{wt} | | TCA ACG TTA GCT TCA | (SEQ ID NO:1) | |
| | | | CC | | |
| c-myc anti-gene | PNA-myc_{wt}-R | (R) | TCA ACG TTA GCT TCA | (SEQ ID NO:1) | |
| rhodaminated | | | CC | | |
| c-myc anti-gene link | PNA-myc_{wt}-T | | TCA ACG TTA GCT TCA | (SEQ ID NO:1) | T |
| | | | CC | | |
| c-myc anti-gene | PNA-myc_{wt}-TR | R | TCA ACG TTA GCT TCA | (SEQ ID NO:1) | T |
| rhodaminated | | | CC | | |
| and linked to | | | | | |
| dihydrotestosterone | | | | | |
| c-myc anti-gene | PNA-myc_{wt}- | | TCA ACG TTA GCT TCA | (SEQ ID NO:1) | NLS |
| linked to a Nuclear | NLS | | CC | | |
| Localization | | | | | |
| Signal peptide | | | | | |
| c-myc anti-gene | PNA-mycₘᵤₜ | | TTA ACG CTA GCT TTA | (SEQ ID NO:2) | |
| modified by a | | | CC | | |
| 3-point mutation | | | | | |
| rhodaminated c-myc | PNA-mycₘᵤₜ-R | R | TTA ACG CTA GCT TTA | (SEQ ID NO:2) | |
| anti-gene, modified | | | CC | | |
| by | | | | | |
| a 3-point mutation | | | | | |
| c-myc anti-gene | PNA-mycₘᵤₜ-T | | TTA ACG CTA GCT TTA | (SEQ ID NO:2) | T |
| modified | | | CC | | |
| by a 3-point | | | | | |
| mutation | | | | | |
| linked to | | | | | |
| dihydrotestosterone | | | | | |
| Rhodaminated 3- | PNA-mycₘᵤₜ₋TR | R | TTA ACG CTA GCT TTA | (SEQ ID NO:2) | T |
| point | | | CC | | |
| mutation c-myc | | | | | |
| anti-gene | | | | | |
| linked to | | | | | |
| dihydrotestosterone | | | | | |

As used herein, R represents Rhodamine, T represents dihydrotestosterone and NLS represents PKKKRKV (SEQ ID NO:3).

As used herein, R represents Rhodamine, T represents dihydrotestosterone and NLS represents PKKKRKV (SEQ ID NO:3).

As a positive control, we also tested the wild-type PNAmyc sequence (PNAmyc_{wt}) coupled to the SV40 Nuclear Localization Signal peptide (NLS) PKKKRKV (SEQ ID NO:3) (22), which we already showed allows the anti-gene PNAmyc_{wt} to penetrate intact cell nuclei and efficiently downregulate c-myc overexpression in Burkitt's lymphoma cells (23).

PNA Synthesis

All PNAs used in this work (Table 1) were manually synthesized using a standard method of solid phase peptide synthesis that follows the Boc strategy (24, 25) with minor modifications to allow PNA rhodamination. The synthesis procedures are summarized below:

Twenty-five (25) nmoles of 4-Methyl-benzhydril-amine-Polystyrene-Gly-Boc deprotected resin (Novabiochem AG, Laufelfingen, Switzerland) was treated for 20 min at 40° C. with a coupling reaction mixture containing 4.5 equivalents (eq) of O-(7-azabenzotriazol-1-yl)-1,1,3,3-tetramethyl-uronium hexafluorophosphate (HATU; Perseptive Biosystems, PRIMM, Milan, Italy), 5 equivalents of N,N-diisopropylethylamine (DIPEA; Fluka Chemie AG, Buchs, Switzerland), 7.5 equivalents of Sim-Collidine (Fluka) with the addition of 5 equivalents of α-Boc-Lysine-(ε-Fmoc)-OH (Novabiochem) for rhodaminated PNAs or alternatively 5 equivalents of α-Boc-Lysine-(ε-Z)-OH for non-rhodaminated constructs at 0.1 M final concentration in anhydrous N-methyl Pyrrolidone (NMP; Fluka). Once lysine was coupled to the resin, the Fmoc protected E amino group was selectively deprotected by a 20 min reaction with 20% Piperidine (Fluka) in anhydrous Dimethylformamide (DMF; Fluka). Another treatment with the above coupling mixture with the addition of 5 equivalents of Rhodamine B (Sigma) was then performed to obtain a selective rhodamination of the lysine's ε amino group. Then, in the assembling of the PNA constructs, the linker (Boc-8-amino-3,6 dioxa-octanoyl acid, PRIMM) was first added and then the Boc-PNA monomers (PRIMM) according to the PNA sequence. After the synthesis of the PNA plus one linker was completed, a small portion of the product assembled on the resin was deprotected, cleaved and purified to determine the mass spectrum. A second linker was added to the bulk of the PNA still linked to the resin and followed by the addition of 4,5-Dihydrotestosterone hemisuccinate (≧98% purity, Fluka). All the reactions described were performed in the same coupling mixture after deprotection of Boc-α-amino groups, following standard Trifluoroacetic Acid (TFA) procedure, to form a pseudo-peptidic bond.

PNA Characterization and Purification

All synthesized compounds were analyzed and purified by reverse phase high performance liquid chromatography (RP-HPLC). The analysis of each crude product was performed using a HP 1090 HPLC equipped with a Waters C₁₈ μBondapack column (3.9×300 mm), while the purification was obtained on a Shimadzu LC-8A preparative HPLC equipped with a Waters C₁₈ μBondapack column (19×300 mm). For both analyses the solvent gradient program started with 100% solvent A (0.1% TFA in water) for 5 min; solvent B (0.1% TFA in acetonitrile) was then added with a linear increase up to 40% in 30 min and subsequently up to 100% B in 5 min. The column elutes were monitored with a diode array detector set at 260 nm. Fractions containing the purified products were pooled, vacuum dried, resuspended in TFA, and precipitated with ice-cold diethyl ether. Mass spectra of each compound were taken using a single quadrupole HP Engine 5989-A equipped with an electrospray ion source (ESMS) and set in the positive ion mode.

Cell Culture

Two prostatic carcinoma derived cell lines were used (LNCaP and DU145 cultured in RPMI-1640, 10% charcoal-stripped fetal calf serum). Their features and culture conditions were as previously detailed (26, 27). PNA stock solutions (500 μM in H₂O neutralized with NaOH) were added as an aliquot to the culture medium to their final concentration. For PNA cellular localization experiments, cells were cultured for 24 h in the presence of 2 μM PNAmyc_{wt}-R or PNAmyc_{wt}-TR, their maximal soluble concentration in rhodaminated conditions. In the gene regulation experiments, PNAmyc_{wt}, PNAmyc_{wt}-T, PNAmycₘᵤₜ-T, and PNAmyc_{wt}-NLS were present at a final 10 μM concentration in the culture medium, for 0, 24, 48, or 72 hours. Cell growth was measured by total cell counts from three replicate flasks (1.3-5.0×10⁶ per each 75 cm²/flask) while cell viability was determined by Trypan blue exclusion. The standard deviation was calculated both for cell growth and viability keeping into account the data from all the experiments (minimum of three each).

Cell Fixation, Staining, and Confocal Microscopy

In three identical experiments, cells were washed and resuspended in PBS at a final concentration of 10⁶ cells per ml after 24 hours of exposure to PNAmyc_{wt}-R and PNAmyc_{wt}-TR. Then the cells were fixed by addition of an equal volume of 4% paraformaldehyde at 0° C. for 20 minutes (28), centrifuged and resuspended in MOWIOL (Calbiochem-Novabiochem, San Diego, Calif.) at a concentration of 1.6×10⁴ cells per μl. The cell suspension was spotted on a glass slide in two aliquots of 10 and 5 μl, covered and sealed. Confocal microscopy was performed with a BioRad MRC-1024 Confocal Laser Scanning System equipped with a Zeiss Axioskope (BioRad, Microscopy Division, Hertfordshire, UK). BioRad Laser Scharp Graphic was interfaced for image acquisition using COMOS BioRad software. The laser filter was yellow (568 nm) and the objective magnification X100. Images were acquired in 0.36 μm layers, both in fluorescence and “phase contrast” mode.

Western Blot Analysis

Total cellular proteins samples were derived from 10⁷ cells solubilized in 200 μl Urea lysis buffer (9M Urea, 50 mM Tris, pH 7.0) with brief sonication at 0° C. as needed. Western blot analysis for MYC expression was performed as described previously (29). Briefly, total cellular proteins were electrophoretically separated using 10% acrylamide, 0.4% (w/v) SDS gels and then transferred to a nitrocellulose membrane (Hybond C-extra, Amersham). The membrane was then cut at a 45-50 kb molecular weight level (using as reference Kaleidoscope pre-stained standards, BioRad). Both membrane halves were incubated in parallel overnight with a primary antibody (top with anti-myc antibody, 9E10 Calbiochem, San Diego, Calif.; bottom with anti-H2b antibody kindly provided by Dr. M. Romani, IST, Genova, I). The membranes were washed and exposed to a rabbit anti-mouse IgG (Dakopatts, Glostrup, DK) at room temperature for 1 h. MYC and H2b bands were visualized by the incubation of the membrane with alkaline phosphatase-conjugated goat anti-rabbit IgG (Sigma) at room temperature for 2 h followed by exposure to the 5-bromo-4-chloro-3-indolylphosphate/nitro blue tetrazolium substrate developer (BCIP/NBT, Sigma).

The stained and reassembled Western blot images, from triplicate experiments, were digitally acquired, elaborated in Photoshop, and then quantified using Scion-Image. MYC concentrations were normalized to the matching histone H2b concentrations (a protein that remains constant throughout the treatment described). Therefore MYC concentration is reported as a ratio to an H2b standard. For Western blot analyses, the average error was 14.0% (range 8.3 to 25.1%) in the LNCaP experiments and for those with DU145 averaged 10.2% (range 8.5 to 20.3%).

PNA Design and Synthesis

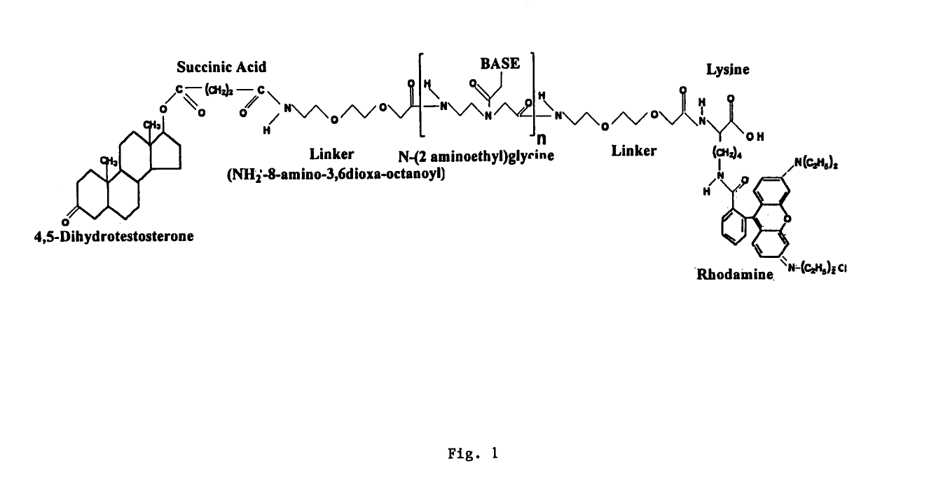

The structure of a PNA modified at its C-terminal by rhodamination and at its N-terminal by the addition of dihydrotestosterone vector (T) is shown in FIG. 1. The reaction of Rhodamine's carboxyl group with the amino group of the C-terminal lysine did not alter the fluorescence of Rhodamine (R), thus allowing detection of PNA distribution in the cell. In the more complex constructs, the PNA component was deliberately spaced at a distance from both the T vector and the R fluorophore in order to assure unimpeded base pair matching of the PNA anti-gene to the complementary sequence of the target c-myc gene. The spacing was obtained by the addition of an octanoyl linker to the N- and C-termini of the PNA. The hemisuccinate derivative of dihydrotestosterone (T) was linked by its carboxyl group to the N-terminal group of the PNA molecule. Furthermore, the succinic acid extension of the vector acts as an additional spacer that may give improved flexibility to the construct and enhance the steric freedom needed for dihydrotestosterone to interact with the androgen receptor in the nucleus of the target cells.

The syntheses of the PNA constructs listed in Table 1 had final yields of 40-50%. After preparative RP-HPLC the purity of each product ranged between 90-95%. In all cases, the mass spectra of the purified PNAs showed [M+H]+ ions values consistent with the molecular weights of the expected molecules. In the specific case of the PNAs liked to dihydrotestosterone in N-terminal position the mass spectra acquired both prior and after the dihydrotestosterone coupling reaction confirmed the correct assembly of the PNA constructs.

PNA and PNA Constructs

Table 1 contains a description of the experimental PNAs used in this study, including their modifications and corresponding abbreviations. In particular, PNA-myc_{wt} is a 17-mer PNA anti-gene for the unique sequence of c-myc, bases 4528-4544, in its II exon (21). The addition of R to PNA and to any of its constructs resulted in a marked decrease in their solubility in the cell culture medium. The maximum solubility of PNA-myc_{wt}-R and PNA-myc_{wt}-TR in RPMI complete medium was only 2 μM; any attempt to increase it resulted in precipitation. All the other PNAs listed in Table 1 had solubilities in excess of 500 μM (stock solution described above), far above the 10 μM concentration used in most experiments.

Cell and Nuclear-Specific Targeting by PNA Constructs

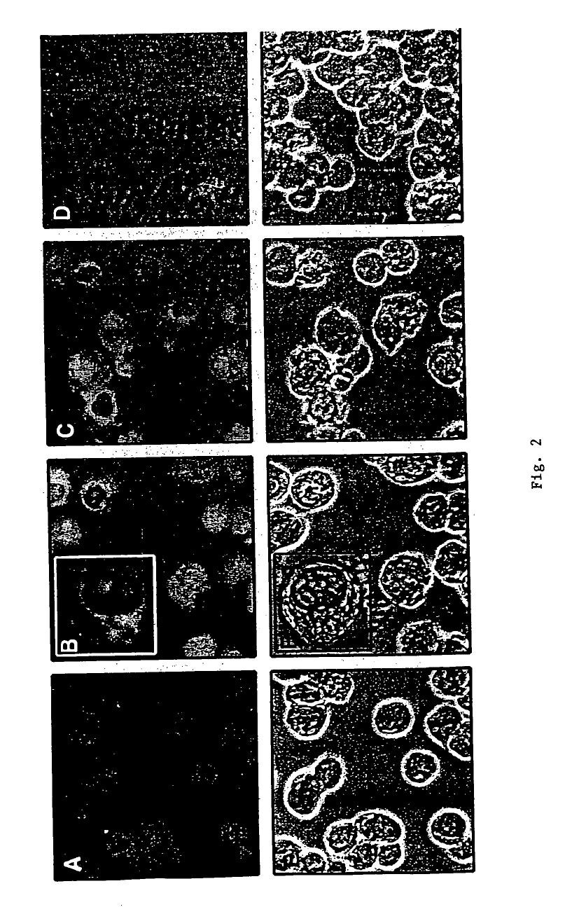

To test whether the covalent attachment of the T vector to PNAs allowed their binding to the AR and transport into the nuclei of intact cells, LNCaP and DU145 cells were exposed to 2 μM PNA-myc_{wt}-R and PNA-myc_{wt}-TR and cultured for 5, 10, and 24 hours and the cells was analyzed using confocal fluorescence and phase contrast microscopy. Although intracellular fluorescence was already detectable after 5 hours, the maximum intensity was obtained at 24 hours. The confocal fluorescence results are shown in FIGS. 2A, 2B, 2C, and 2D, and the phase contrast microscopy results are shown in FIGS. 2E, 2F, 2G and 2H. Specifically, FIGS. 2A and 2E show the cellular localization of PNA-myc_{wt}-R in LNCaP. FIGS. 2C and 2G show the cellular localization of PNA-myc_{wt}-R in DU145 cells.

In LNCaP cells exposed to the vector-free PNA-myc_{wt}-R, the fluorescence had a cytoplasmic localization as shown in FIGS. 2A and 2E. Moreover, cytoplasmic and cellular localization of PNA-myc_{wt}-TR in LNCaP cells are shown in FIGS. 2B and 2F, and PNA-myc_{wt}-TR was clearly detectable in the cell nuclei. Untreated cells, however, showed only background intracellular fluorescence after 24 hours.

In DU145 cells both PNA-myc_{wt}-R (FIGS. 2C and 2G) and PNA-myc_{wt}-TR (FIGS. 2D and 2H) fluorescence was entirely cytoplasmic. Notably, this fluorescence in DU145 cells was far weaker in the construct carrying the T vector than in the matching PNA-myc_{wt}-R. Without being limited to any particular theory of operation, it is believed that this effect was due to the lack of androgen receptors in DU145 cells combined with the bulkiness and low solubility of the PNA-myc_{wt}-TR construct. Thus, the T vector met two criteria for effective PNA delivery, namely recognition of cell phenotype and transport into the nucleus. PNA cellular localization was also quantified, not only in the final images, but also on all the sections of the confocal acquisitions. Differences were significant when the optical sections passing through the middle of the nuclei (23) were evaluated (data not shown).

Effects of PNAs on Cell Growth

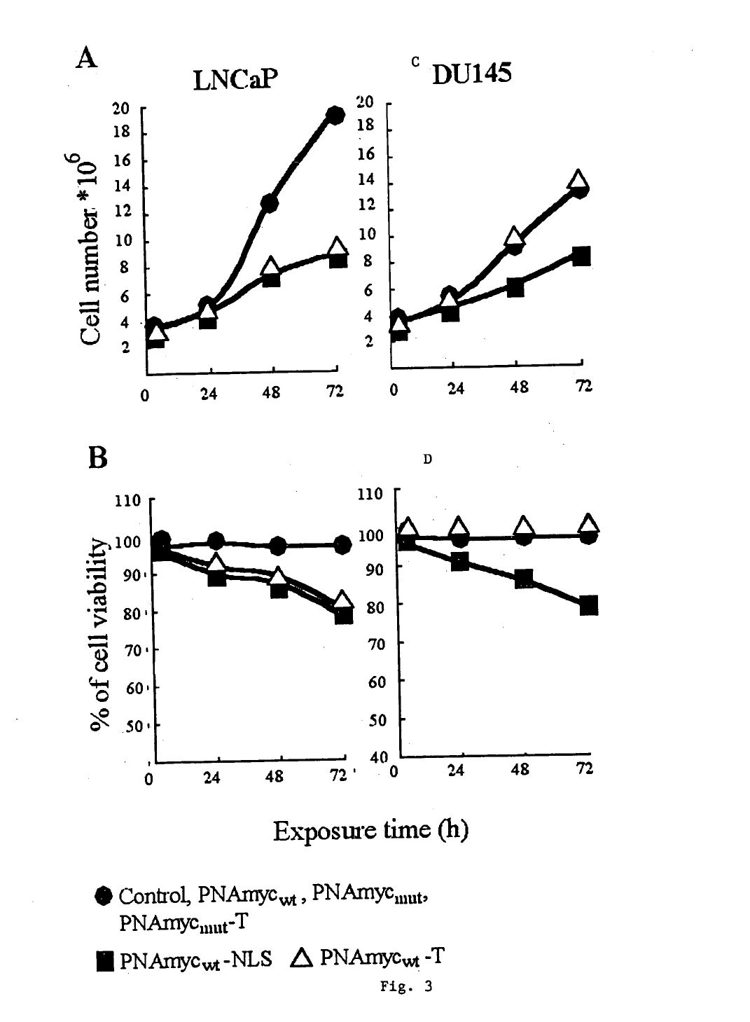

LNCaP and DU145 cells were treated with a 10 μM concentration of PNA constructs in the culture medium (PNA-myc_{wt}-T (Δ), PNA-myc_{wt}, PNA-mycₘᵤₜ, PNA-mycₘᵤₜ-T (●) as negative controls and PNA-myc_{wt}-NLS (▪) as positive control). Cell numbers (FIGS. 3A and C) and viability (FIGS. 3B and D) were estimated, as described above, at increasing times of exposure to different PNA constructs.

The PNA concentration of 10 μM in the cell medium was chosen since we have previously proven that in similar experimental conditions this PNA-myc_{wt}-NLS concentration caused maximum inhibition of MYC expression with a small decrease in cell viability (23).

In LNCaP cells, exposure to PNA-myc_{wt}-T and to PNA-myc_{wt}-NLS caused a time-dependent decrease in cell growth that became as low as 34% of control at 72 hours. Treatment with any of the other non-rhodaminated PNAs listed (Table 1), other than PNA-myc_{wt}-T and PNA-myc_{wt}-NLS, resulted in cell growth rates essentially superimposable to those of untreated cells as shown in FIG. 3A.

In DU145 cells only, the construct PNA-myc_{wt}-NLS was used as a positive control for nuclear localization and resultant c-myc gene down-regulation. As previously shown, this nuclear localization signal peptide effectively delivers PNA to cells in a specific way (23), resulting in a time-dependent inhibition of cell growth of 36% at 72 h. The standard deviation for triplicate experiments is not illustrated in the graphs since its range is only between 1.1% and 5.8% for LNCaP cells and 1.3% and 6.1% for DU145 cells. See FIG. 3C.

Effects of PNAs on Cell Viability

In LNCaP cells, exposure to either PNA-myc_{wt}-T or PNA-myc_{wt}-NLS caused a modest time-dependent decline in cell viability that reached only about 20% after 72 h of treatment (FIG. 3B). An effect on the viability of the same magnitude and timing was also observed in DU145 cells (FIG. 3D), but only when treated with PNA-myc_{wt}-NLS. In both cell lines, exposure to any of the (non-rhodaminated) PNAs had no effect on viability as compared to untreated cells. The standard deviation was not illustrated graphically as it ranged only between 2% and 6% both for LNCaP and DU145 cell lines.

Modulation of c-myc Expression by PNAs

LNCaP and DU145 cells were treated for the indicated time periods with 10 μM concentration of PNA-myc_{wt}-T (Δ), PNA-myc_{wt}, PNA-mycₘᵤₜ, PNA-mycₘᵤₜ-T (●), and PNA-myc_{wt}-NLS (▪). As shown in Fugure 4A, the amount of MYC protein was evaluated by Western blot analysis of total nuclear proteins. The effects of PNAs on expression of the c-myc gene in LNCaP and DU145 cells were also obtained by immunochemical measurement of the MYC protein content of cell lysates, using data obtained from at least a triplicate run of each set of immunostained Western blots. In each case, the MYC content of the whole cell lysate was compared to that of histone H2b, a protein that remains constant throughout the treatment described. The constancy of H2b made it possible to represent variations in MYC protein concentration normalized to the H2b content in the same lysate.

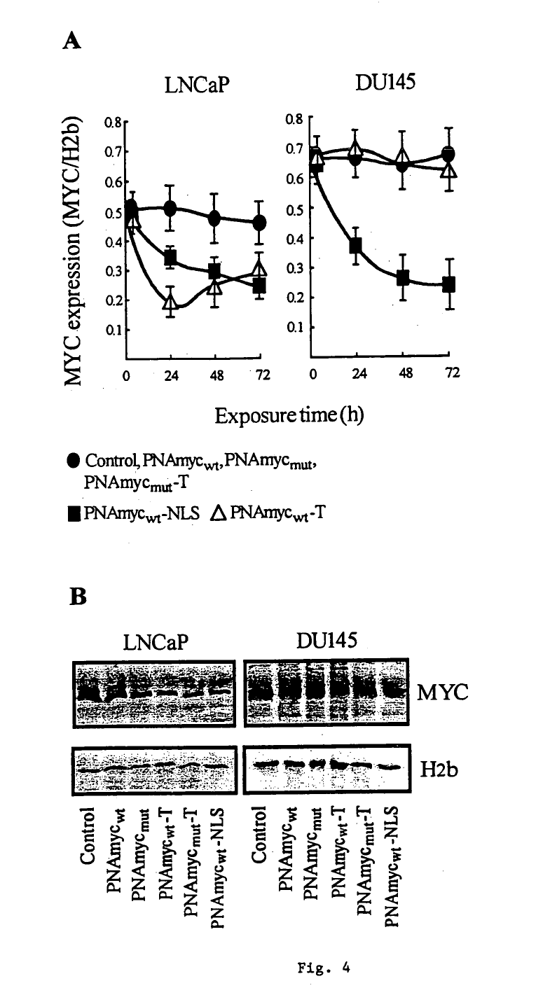

Data are shown in FIG. 4A as the ratio between the intensity of MYC and H2b. The standard deviations are also illustrated. The pictures of Western bands for MYC and H2b at the 24 hours exposure time-point are shown in FIG. 4B. Then the effects of the PNAs on MYC expression were compared wherein parallel cultures of LNCaP and DU145 cells were exposed to 10 μM each of the non-rhodaminated PNAs (listed in Table 1) for 0, 24, 48, or 72 hours. Under these conditions, a time-dependent decrease in MYC content was observed in LNCaP cells, but only by treatment with PNAs covalently linked to vectors: PNA-myc_{wt}-T and PNA-myc_{wt}-NLS. In both cases, the decrease was time-dependent, yet different for the two vectors. Whereas PNA-myc_{wt}-NLS induced a linear decrease in MYC content (up to 52% at 72 h), PNA-myc_{wt}-T caused a maximal effect (−63%) at 24 h as compared to −50% or −42% at 48 or 72 hours, respectively (see FIG. 4A). In DU145 cells that lack AR, only PNA-myc_{wt}-NLS caused a time-dependent linear inhibition of MYC, reaching about 65% at 72 hours. These results are significant as illustrated by the error bars in FIG. 4A. None of the other listed PNAs had a significant effect.

A similar selective trend of MYC down-regulation by PNA-myc_{wt}-T was observed in preliminary experiments using rhodaminated PNAs when comparing PNA-myc_{wt}-R and PNA-myc_{wt}-TR at a much lower concentration (2 μM) because of limited solubility of these R-PNA constructs. In this case, MYC showed a small selective decrease only in LNCaP cells treated with PNA-myc_{wt}-TR (−20% at 24 hours, −30% at 48 and 72 h: data not shown).

Specificity of PNA in Targeting the c-myc Gene

Of particular significance are the comparative results obtained with PNAs complementary to unique c-myc sequence to those in which that sequence had been altered by a three-point mutation that did not change the ratio of purine to pyrimidine. The significant inhibition of MYC expression by PNAs containing the wild-type sequence is contrasted with the negligible effects of the PNA mutants in both LNCaP and DU145 cells (see FIGS. 4A, B).

In considering the efficient application of PNAs as anti-gene and antisense agents, the ability to target a particular cell type is highly advantageous. The experiments described herein, comparing two prostatic cancer cell lines differing in their expression of the AR gene, show that a dihydrotestosterone vector covalently linked to a PNA anti-myc gene discriminated between those cell types based on the presence or absence of the AR receptor.

In conclusion, the data indicate that anti-gene PNAs selectively bind and down-regulate the complementary sequences in the target gene when linked to specific hormonal vectors. These vectors appear to facilitate the uptake of PNA into the nucleus of living cells that contain their cognate hormone receptor, findings suggestive of vector-enhanced nuclear translocation mediated by AR.

By way of additional examples, the following PNAs may be prepared following the teachings provided herein, namely: (1) cagctggaattcggggc (SEQ ID NO:4); (2) cggggcttaaggtcgac (SEQ ID NO:5); (3) ccgtccaagacctacc (SEQ ID NO:6); (4) ccatgttttgccatt (SEQ ID NO:7); and (5) gacagtgtcacacatt (SEQ ID NO:8). The first two PNA sequences hybridize with a portion of the human STAT3 (signal transducers and activators of transcription) gene, which are expressed in hormone-dependent and hormone-independent cells such as mammary, prostate, pituitary, brain, gonad, tissues of the reproductive system, skin, immune cells, blood, bone, thyroid, liver, thymus etc. It is not ubiquitously expressed in all cells, however. These cells can be targeted by coupling the PNAs to a ligand that binds a nuclear receptor produced by any one or more of these cell types. The resulting compositions are used to treat diseases such as cancer characterized by or involving STAT3 over-expression. The third, fourth and fifth PNA sequences bind to a portion of the human androgen receptor gene. They may be targeted to androgen receptor-containing cells using a ligand that binds to a different nuclear receptor so as to treat prostate cancer, BPH, male pattern baldness and tumors arising from androgen-dependent metastasis.

Citations of Publications Referenced herein:

1. Egholm, M., et al. (1992) “Peptide Nucleic Acids (PNA): Oligonucleotide analogues with an achiral peptide backbone,” J. Am. Chem. Soc., 114: 1895-1897;

2. Nielsen, P. E., et al. (19.91) “Sequence-selective recognition of DNA by strand displacement with a thymine-substituted polyamide,” Science, 254: 1497-1500;

3. Egholm, M., et al. (1993) “PNA hybridizes to complementary oligonucleotides obeying the Watson-Crick hydrogen-bonding rules,” Nature, 365: 566-568;

4. Almarsson, O., et al. (1993) “Molecular mechanics calculations of the structures of polyamide nucleic acid DNA duplexes and triple helical hybrids,” Proc. Natl. Acad. Sci. U. S. A., 90: 7518-7522;

5. Tomac, S., et al. (1996) “Ionic effects on the stability and conformation of peptide nucleic acids (PNA) complexes,” J. Am. Chem. Soc., 118: 5544-5552;

6. Bentin, T., and Nielsen, P. E. (1996) “Enhanced peptide nucleic acid binding to supercoiled DNA: possible implications for DNA ‘breathing’ dynamics,” Biochemistry , 35: 8863-8869;

7. Boffa, L. C., et al. (1997) “Contrasting effects of PNA invasion of the chimeric DMMYC gene on transcription of its myc and PVT domains,” Oncology Res., 9: 41-51;

8. Boffa, L. C., et al. (1995) “Isolation of active genes containing CAG repeats by DNA strand invasion by a peptide nucleic acid,” Proc. Natl. Acad. Sci. U.S.A., 92: 1901-1905;

9. Demidov, V. V., et al. (1994) “Stability of peptide nucleic acids in human serum and cellular extracts,” Biochem. Pharmacol., 48: 1310-1313;

10. Boffa, L. C., et al. (1996) “Invasion of the CAG triplet repeats by a complementary peptide nucleic acid inhibits transcription of the androgen receptor and TATA-binding protein genes and correlates with refolding of an active nucleosome containing a unique AR gene sequence,” J. Biol. Chem., 271: 13228-13233;

11. Tyler, B. M., et al. (1999) “Peptide nucleic acids targeted to the neurotensin receptor and administered i.p. cross the blood-brain barrier and specifically reduce gene expression,” Proc. Natl. Acad. Sci. U.S.A., 96: 7053-7058;

12. Gray, D. G, et al. (1997) “Transformed and immortalized cellular uptake of oligodeoxynucleoside phosphorotioathes, 3′alkylamino oligodeoxynucleotides,2′-O-methyl oligo-ribonucleotides, oligodeoxynucleosides methylphosphonates, and peptide nucleic acids,” Biochem. Pharmacol., 53: 1465-1476;

13. Bonham, M. A., et al. (1995) “An assessment of the antisense properties of RNase H-competent and steric-blocking oligomers,” Nucleic Acids Res., 23: 1197-1203;

14. Pooga, M., et al. (1998) “Cell penetrating PNA constructs regulate galanin receptor levels and modify pain transmission in vivo,” Nature Biotech., 16:857-861;

15. Aldrian-Herrada, G., et al. (1998) “A peptide nucleic acid (PNA) is more rapidly internalized in cultured neurons when coupled to a retro-inverso delivery peptide. The antisense activity depresses the target mRNA and protein in magnocellular oxytocin neurons,” Nucleic Acids Res., 26: 4910-4916;

16. Scarfì, S., et al. “Synthesis, uptake, and intracellular metabolism of a hydrophobic tetrapeptide-peptide nucleic acid (PNA)-biotin molecule,” Biochem. Biophys. Res. Commun., 236: 323-326;

17. Branden, L. J., et al. (1999) “A peptide nucleic acid nuclear localization signal fusion that mediates nuclear transport of DNA,” Nature Biotech., 17: 784-787;

18. Tilley, W. D., et al. (1990) “Androgen Receptor gene expression in human prostate carcinoma cell lines,” Cancer Res., 50: 5382-5386;

19. Trapman, J., and Brinkmann, A. O. (1996) “The Androgen Receptor in prostate cancer,” Pathol. Res. Pract., 192: 752-760;

20. Balaji, K. C., et al. (1997) “Antiproliferative effects of c-myc antisense oligonucleotide in prostate cancer cells: a novel therapy in prostate cancer,” Urology, 50: 1007-1015;

21. Grazin, C., et al. (1984) “Nucleotide sequence of the human c-myc locus: provocative open reading frame within the first exon,” EMBO J., 3: 383-387;

22. Lanford, R. E., et al. (1986) “Induction of nuclear transport with a synthetic peptide homologous to the SV40 T antigen transport signal,” Cell, 46:575-582;

23. Cutrona, G., et al. (2000) “Effects in live cells of a c-myc anti-gene PNA liked to a nuclear localization signal,” Nature Biotechnology,18: in press;

24. Merrifield, B. (1997) “Concept and early development of solid-phase peptide synthesis,” Methods Enzymol., 289: 3-13;

25. Christensen, L., et al. (1995) “Solid-phase synthesis of peptide nucleic acids,” J. Pept. Sci., 3:175-183;

26. Horoszewicz, J. S, et al. (1983) “LNCaP model of human prostatic carcinoma,” Cancer Res., 43: 1809-1818;

27. Mickey, D. D., et al. (1977) “Heterotransplantation of a human prostatic adenocarcinoma cell line in nude mice,” Cancer Res., 37: 4049-4058;

28. Curtis Bird, et al. (1994) In J. E: Celis (ed.), Cell Biology: A Laboratory Handbook, Vol. 1, pp.278 New York, Academic Press;

29. Cutrona, G., et al. (1995) “Transfection of the c-myc oncogene into normal Epstein-Barr virus-harboring B cells results in new phenotypic and functional features resembling those of Burkitt Lymphoma cells and normal centroblasts,” J. Exp. Med., 181: 699-711.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated as being incorporated by reference herein.

Those skilled in the art will recognize, or be able to ascertain, using no more than routine experimentation, numerous equivalents to the specific substances and procedures described herein. Such equivalents are considered to be within the scope of this invention, and are covered by the following claims.

1. A composition of matter comprising a peptide nucleic acid (PNA) linked to a ligand that binds a nuclear receptor.

2. The composition of claim 1 wherein said ligand binds a nuclear receptor which is an intracellular steroid receptor.

3. The composition of claim 1 wherein the ligand comprises a sterol.

4. The composition of claim 3 wherein the sterol comprises cholesterol.

5. The composition of claim 1 wherein said ligand binds a nuclear receptor which is an intracellular hormone receptor.

6. The composition of claim 1 wherein said ligand binds a nuclear receptor which is an intracellular non-hormone receptor.

7. The composition of claim 1 wherein said ligand binds a nuclear receptor which is an intracellular non-androgen hormone receptor.

8. The composition of claim 1 wherein said ligand comprises an androgen.

9. The composition of claim 8 wherein said androgen is testosterone or dihydrotestosterone.

10. The composition of claim 8 wherein said androgen comprises 7 α-methyl-19-nortestosterone (MENT).

11. The composition of claim 1 wherein the ligand comprises an estrogen.

12. The composition of claim 1 wherein the ligand comprises a progestin.

13. The composition of claim 12 wherein said progestin is progesterone.

14. The composition of claim 1 wherein said ligand binds a nuclear receptor which is a vitamin receptor.

15. The composition of claim 1 wherein said ligand comprises a glucocorticoid.

16. The composition of claim 1 wherein said ligand comprises a retinoid.

17. The composition of claim 1 wherein said ligand comprises a mineralocortoid.

18. The composition of claim 1 wherein said PNA binds a c-myc gene or a portion thereof sufficient to inhibit transcription of said gene.

19. The composition of claim 18 wherein said PNA comprises a sequence TCA ACG TTA GCT TCA CC (SEQ ID NO:1).

20. The composition of claim 1 further comprising a detectable label.

21. The composition of claim 1 formulated for oral, topical, nasal or parenteral administration.

22. A method of introducing a PNA into a cell nucleus, comprising contacting the cell with the composition of claim 1.

23. The method of claim 22 the cell is a mammalian cell.

24. The method of claim 23 wherein the composition is administered to a mammal.

25. The method of claim 25 wherein the mammal is a human.

26. The method of claim 22 wherein the cell is a cancer cell.

27. The method of claim 22 wherein the cell is a prostate, mammary, ovarian, uterine, cervical, skin, brain, neural, thyroid, kidney, adrenal, pancreatic, colonic, lung, blood or a B-cell.

28. The method of claim 24 wherein the cell is infected with a virus.

29. A method of making a composition comprising a PNA linked to a ligand that binds a nuclear receptor, comprising preparing the PNA, preparing the ligand, and linking the PNA and the ligand.
